(19) United States
(12) Patent Application Publication
(10) Pub. No.: US 2007/0275994 A1
(43) Pub. Date: Nov. 29, 2007

(54) Novel Antinfective Compounds

(76) Inventors: Braj Bhushan Lohray, Ahmedabad (IN); Vidya Bhusha Lohray, Ahmedabad (IN); Brijesh Kumar Srivastava, Ahmedabad (IN)

Correspondence Address: NIXON & VANDERHYE, PC, 901 NORTH GLEBE ROAD, 11TH FLOOR, ARLINGTON, VA, 22203, US

(21) Appl. No.: 10/559,628

(22) PCT Filed: Jun. 4, 2004
(86) PCT No.: PCT/IN04/00155

§ 371 Date: Jun. 27, 2007

(30) Foreign Application Priority Data
Jun. 6, 2003 (IN) 582/MUM/2003

(51) Int. Cl. A61K 31/4365; A61K 31/4355; A61P 31/00; C07D 471/04

(52) U.S. Cl. 514/301; 514/302; 546/114; 546/115

(57) ABSTRACT

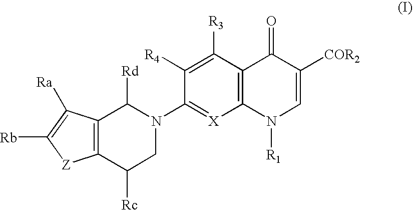

The present invention describes novel antiinfective compounds of formula (I), process for their preparation and pharmaceutical compositions containing them.

### FIELD OF INVENTION

The present invention relates to novel compounds of general formula (I), their analogs, their stereoisomers, tautomeric forms, novel intermediates involved in their synthesis, their pharmaceutically acceptable salts and pharmaceutical compositions containing them. The present invention also relates to a process of preparing compounds of general formula (I), their analogs, their derivatives, their stereoisomers, their tautomeric forms, their pharmaceutically acceptable salts, pharmaceutical compositions containing them, and novel intermediates involved in their synthesis.

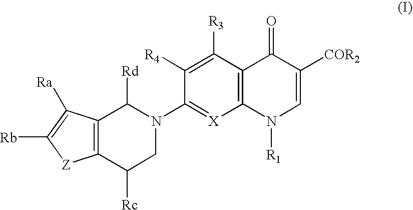

### BACKGROUND TO THE INVENTION

Antibiotic resistance is a serious concern worldwide as it would result in strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity. The compounds of the present invention though being primarily effective against Gram-positive pathogens are also effective against certain Gram-negative pathogens.

Gram-positive pathogens, for example Staphylococci Enterococci, Streptococci and Mycobacteria, are particularly important because of the development of resistant strains which are both difficult to treat and difficult to eradicate from the hospital environment once established. Examples of such strains are methicillin resistant staphylococcus (USA), methicillin resistant coagulase negative staphylococci (MRCNS), penicillin resistant Streptococcus pneumoniae and multiply resistant Enterococcus faecium, community acquired pathogens (CAP), and so on.

Quinolones as a class of antibacterial agents are well known and are being used extensively throughout the world. They are potent inhibitors of Gram positive as well as Gram negative pathogens and may be administered orally or intravenously. A wide range of quinolone antibacterials have been introduced in the last decade which includes norfloxacin, ciprofloxacin, ofloxacin, and the recently launched gatifloxacin and moxifloxacin. However, some of the quinolone antibacterials have been associated with significant side effects (J. Antimicrob. Chemother., 1994; 33: 685) and some of them have been discontinued at different stages of development (e.g. Trovafloxacin).

The quinolones inhibit bacterial growth by inhibition of DNA gyrase topoisomerase II and topoisomerase IV (Gootz, Medicinal Research, 1996; Rev. 16:433). The gyrase interaction appears to rely on the N-carbonyl-carboxyl relationship at the C-7 position in the quinoline nucleus in this class of compounds. Quinolone antibacterials and their methods of preparation has been described in WO 0296908, WO 0153273, WO 0132655, WO 0129035, WO 9640190, WO 9640156, WO 9602540, U.S. Pat. No. 4,994,599, U.S. Pat. No. 4,990,517, U.S. Pat. No. 4,980,470, U.S. Pat. No. 4,980,373, U.S. Pat. No. 4,945,160, U.S. Pat. No. 4,954,507, U.S. Pat. No. 4,880,814, U.S. Pat. No. 4,795,751, U.S. Pat. No. 4,670,444, OS 3816119, EP 0805156, EP 0421668, EP 0449445, EP 0300311, EP 0241206, EP 0235762, EP 0167763, EP 0155006, EP 0140116, EP 0028698, DE 3441788, DE 3519286, which are incorporated herein as reference in their entirety.

However, due to increase in antibacterial resistance and also otherwise there is a continuing need for discovering compounds which are more effective against resistant bacteria, have improved intestinal absorption, metabolic stability, and exhibit less toxicity.

### OBJECTIVES

The main objective of the present invention thus is to provide novel compounds of general formula (I), their analogs, their stereoisomers, their tautomeric forms, novel intermediates involved in their synthesis, their pharmaceutically acceptable salts, their pharmaceutically acceptable solvates and pharmaceutical compositions containing them or their mixtures suitable in the treatment of infectious diseases.

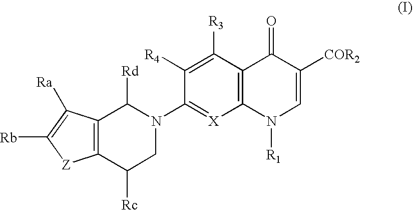

Another objective of the present invention is to provide a process for the preparation of novel compounds of general formula (I), their analogs, their stereoisomers, their tautomeric forms, novel intermediates involved in their synthesis, their pharmaceutically acceptable salts, pharmaceutically acceptable solvates and pharmaceutical compositions containing them.

Yet another objective of the present invention is to provide pharmaceutical compositions containing compounds of general formula (I), their analogs, their stereoisomers, their tautomeric forms, their pharmaceutically acceptable salts, solvates and their mixtures having pharmaceutically acceptable carriers, solvents, diluents, excipients and other media normally employed in their manufacture.

Still another objective of the present invention is to provide a method of treatment of antibiotic resistant pathogens, by administering a therapeutically effective & non-toxic amount of the compound of formula (I) or their pharmaceutically acceptable compositions to the mammals.

### SUMMARY OF THE INVENTION

The present invention describes a group of novel compounds useful as antibacterial agents. The novel compounds are defined by the general formula (I) below:

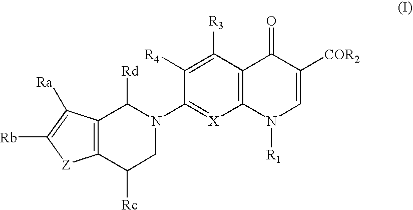

The compounds of the present invention are useful in the treatment of the human or animal body, as preventives and therapeutic agents for infectious diseases. The compounds of this invention have excellent antimicrobial action against various human and veterinary pathogens including but not limited to multiply-resistant staphylococci and streptococci, as well as anaerobic organisms including those of the bacteroides and clostridia species, and acid-fast Mycobacterium tuberculosis and Mycobacterium avium with better efficacy, potency and minimum toxic effects.

### DETAILED DESCRIPTION OF THE DESCRIPTION

The novel compounds of the present invention are defined by the general formula (I) below: 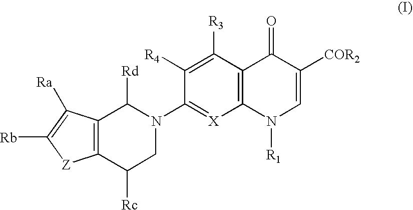
wherein

R₁ represents hydrogen, linear or branched, substituted or unsubstituted groups selected from (C₁-C₁₂) alkyl, (C₂-C₁₂)alkenyl, (C₂-C₂)alkynyl, (C₃-C₁₂)cycloalkyl; substituted or unsubstituted groups selected from aryl, heteroaryl or heterocyclic groups;

R₂ is selected from hydrogen, —OBF₂ or —OR₆,

Where R₆ represents hydrogen, (C₁-C₆)alkyl, (C₃-C₆)alkenyl or (C₃-C₆)alkynyl groups, which may optionally be substituted;

R₃ represents H, OH, linear or branched, substituted or unsubstituted groups selected from —O(C₁-C₁₂) alkyl, —O(C₂-C₁₂) alkenyl, —O(C₂-C₁₂) alkynyl, halo, NO₂, CN, or NR′R″ groups, where R′R″ may be same or different and independently represent H, linear or branched, substituted or unsubstituted groups selected from (C₁-C₆)alkyl, (C₂-C₆) alkenyl, (C₂-C₆)alkynyl or acyl groups;

R₄ represents H or halogen atom;

X represents N or C—R₇,

where R₇ represents H, —OH, —(O)ₙ(C₁-C₆) substituted or unsubstituted alkyl where n is 0 or 1, —NO₂, —NH₂, —NHCOCH₃, —CN, —COOH groups;

R₁ and R₇ can be taken together with the atoms to which they are attached to form a cyclic ring, which may optionally be substituted and may also optionally contain from 1 to 3 heteroatoms selected from O, N and S;

Ra, Rb may be same or different and represents hydrogen, halogen, haloalkyl, perhaloalkyl, haloalkoxy, perhaloalkoxy, hydroxy, thio, amino, nitro, cyano, formyl, or substituted or unsubstituted groups selected from linear or branched (C₁-C₁₂)alkyl, linear or branched (C₁-C₁₂)alkenyl, linear or branched (C₁-C₁₂)alkynyl, (C₃-C₇)cycloalkyl, (C₃-C₇)cycloalkenyl, bicycloalkyl, bicycloalkenyl, (C₁-C₁₂)alkoxy, (C₁-C₁₂)alkenoxy, cyclo(C₃-C₇)alkoxy, aryl, aryloxy, aralkyl, ar(C₁-C₁₂)alkoxy, heterocyclyl, heteroaryl, heterocyclyl(C₁-C₁₂)alkyl, heteroar(C₁-C₁₂)alkyl, heteroaryloxy, heteroar(C₁-C₁₂)alkoxy, heterocycloxy, heterocyclylalkyloxy, acyl, acyloxy, acylamino, mono-substituted or di-substituted amino, arylamino, aralkylamino, carboxylic acid and its derivatives such as esters and amides, hydroxyalkyl, aminoalkyl, mono-substituted or di-substituted aminoalkyl, alkoxyalkyl, aryloxyalkyl, aralkoxyalkyl, (C₁-C₁₂)alkylthio, thio(C₁-C₁₂)alkyl, arylthio, (C₁-C₁₂)alkoxycarbonylamino, aryloxycarbonylamino, aralkyloxycarbonylamino, aminocarbonylamino, alkylaminocarbonylamino, alkylamidino, alkylguanidino, dialkylguanidino, hydrazino, alkyl hydrazino, alkoxyamino, hydroxylamino, derivatives of sulfenyl and sulfonyl groups, sulfonic acid and its derivatives, phosphonic acid and its derivatives; Rc & Rd may be same or different and represents hydrogen, substituted or unsubstituted groups selected from alkyl, alkenyl groups;

Z represents O, S or NH. which may optionally be substituted;

The term “substituted” used in combination with other radicals, denotes suitable substituent on that radical such as substituted alkyl, substituted alkenyl, substituted alkynyl, substituted cycloalkyl, substituted aryl, etc, mentioned anywhere in the specification. The suitable substituent include, but are not limited to the following radicals, either alone or in combination thereof such as, hydroxyl, oxo, halo, thio, nitro, amino, cyano, formyl, hydrazino, alkyl, haloalkyl, perhaloalkyl, alkoxy, haloalkoxy, perhaloalkoxy, alkenyl, alkynyl, cycloalkyl, cycloalkenyl, bicycloalkyl, bicycloalkenyl, alkoxy, alkenoxy, cycloalkoxy, aryl, aryloxy, aralkyl, aralkoxy, heterocylyl, heteroaryl, heterocyclylalkyl, heteroaralkyl, heteroaryloxy, heteroaralkoxy, heterocyclyloxy, heterocyclylalkoxy, heterocyclylalkoxyacyl, acyl, acyloxy, acylamino, monosubstituted or disubstituted amino, arylamino, aralkylamino, carboxylic acid and its derivatives such as esters and amides, carbonylamino, hydroxyalkyl, aminoalkyl, alkoxyalkyl, aryloxyalkyl, aralkoxyalkyl, alkylthio, thioalkyl, arylthio, alkoxycarbonylamino, aryloxycarbonylamino, aralkyloxycarbonylamino, aminocarbonylamino, alkylaminocarbonylamino, alkoxyamino, hydroxylamino, sulfenyl derivatives, sulfonyl derivatives, sulfonic acid and its derivatives, phosphonic acid and its derivatives.

When any of the substituents are further substituted, the substituents may be selected from the following radicals, alone or in combination with other radicals, such as, hydroxyl, oxo, halo, thio, nitro, amino, cyano, formyl, amidino, guanidino, hydrazino, alkyl, haloalkyl, perhaloalkyl alkoxy, haloalkoxy, perhaloalkoxy, alkenyl, alkynyl, cycloalkyl, cycloalkenyl, bicycloalkyl, bicycloalkenyl, alkoxy, alkenoxy, cycloalkoxy, aryl, aryloxy, aralkyl, aralkoxy, heterocylyl, heteroaryl, heterocyclylalkyl, heteroaralkyl, heteroaryloxy, heteroaralkoxy, heterocyclyloxy, heterocyclylalkoxy, heterocyclylalkoxyacyl, acyl, acyloxy, acylamino, monosubstituted or disubstituted amino, arylamino, aralkylamino, carboxylic acid and its derivatives such as esters and amides, carbonylamino, hydroxyalkyl, aminoalkyl, alkoxyalkyl, aryloxyalkyl, aralkoxyalkyl, alkylthio, thioalkyl, arylthio, alkoxycarbonylamino, aryloxycarbonylamino, aralkyloxycarbonylamino, aminocarbonylamino, alkylaminocarbonylamino, alkoxyamino, hydroxylamino, sulfenyl derivatives, sulfonyl derivatives, sulfonic acid and its derivatives, phosphonic acid and its derivatives

The term “alkyl” used herein, either alone or in combination with other radicals, denotes a linear or branched radical containing one to twelve carbons, such as methyl, ethyl, n-propyl, iso-propyl, n-butyl, sec-butyl, tert-butyl, amyl, t-amyl, n-pentyl, n-hexyl, iso-hexyl, heptyl, octyl and the like.

The term “alkenyl” used herein, either alone or in combination with other radicals, denotes a linear or branched radical containing two to twelve carbons; such as vinyl, allyl, 2-butenyl, 3-butenyl, 2-pentenyl, 3-pentenyl, 4-pentenyl, 2-hexenyl, 3-hexenyl, 4-hexenyl, 5-hexenyl, 2-heptenyl, 3-heptenyl, 4-heptenyl, 5-heptenyl, 6-heptenyl and the like. The term “alkenyl” includes dienes and trienes of straight and branched chains.

The term “alkynyl” used herein, either alone or in combination with other radicals, denotes a linear or branched radical containing two to twelve carbons, such as ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 3-hexynyl, 4-hexynyl, 5-hexynyl, and the like. The term “alkynyl” includes di- and tri-ynes.

The term “cycloalkyl” used herein, either alone or in combination with other radicals, denotes a radical containing three to seven carbons, such as cyclopropyl, cyclobutyl, cyclopentyl, cyclohexyl, cycloheptyl and the like.

The term “cycloalkenyl” used herein, either alone or in combination with other radicals, denotes a radical containing three to seven carbons, such as cyclopropenyl, 1-cyclobutenyl, 2-cylobutenyl, 1-cyclopentenyl, 2-cyclopentenyl, 3-cyclopentenyl, 1-cyclohexenyl, 2-cyclohexenyl, 3-cyclohexenyl, 1-cycloheptenyl, cycloheptadienyl, cycloheptatrienyl, and the like.

The term “alkoxy” used herein, either alone or in combination with other radicals, denotes a radical alkyl, as defined above, attached directly to an oxygen atom, such as methoxy, ethoxy, n-propoxy, iso-propoxy, n-butoxy, t-butoxy, iso-butoxy, pentyloxy, hexyloxy, and the like.

The term “alkenoxy” used herein, either alone or in combination with other radicals, denotes an alkenyl radical, as defined above, attached to an oxygen atom, such as vinyloxy, allyloxy, butenoxy, pentenoxy, hexenoxy, and the like.

The term “cycloalkoxy” used herein, either alone or in combination with other radicals, denotes a radical containing three to seven carbon atoms, as defined above, attached directly to an oxygen atom, such as cyclopropyloxy, cyclobutyloxy, cyclopentyloxy, cyclohexyloxy, cycloheptyloxy and the like.

The term “halo” or “halogen” used herein, either alone or in combination with other radicals, such as “haloalkyl”, “perhaloalkyl” etc refers to a fluoro, chloro, bromo or iodo group. The term “haloalkyl” denotes an alkyl radical, as defined above, substituted with one or more halogens; such as fluoromethyl, difluoromethyl, trifluoromethyl, fluoroethyl, difluoroethyl, trifluoroethyl, mono or polyhalo substituted methyl, ethyl, propyl, butyl, pentyl or hexyl groups. The term “haloalkoxy” denotes a haloalkyl, as defined above, directly attached to an oxygen atom, such as fluoromethoxy, chloromethoxy, fluoroethoxy chloroethoxy groups, and the like. The term “perhaloalkoxy” denotes a perhaloalkyl radical, as defined above, directly attached to an oxygen atom, trifluoromethoxy, trifluoroethoxy, and the like.

The term “aryl” or “aromatic” used herein, either alone or in combination with other radicals, denotes ah aromatic system containing one, two or three rings wherein such rings may be attached together in a pendant manner or may be fused, such as phenyl, naphthyl, tetrahydronaphthyl, indane, biphenyl, and the like. The term “aralkyl” denotes an alkyl group, as defined above, attached to an aryl, such as benzyl, phenethyl, naphthylmethyl, and the like. The term “aryloxy” denotes an aryl radical, as defined above, attached to an alkoxy group, as defined above, such as phenoxy, naphthyloxy and the like, which may be substituted. The term “aralkoxy” denotes an arylalkyl moiety, as defined above, attached directly to an oxygen atom, such as benzyloxy, phenethyloxy, naphthylmethyloxy, phenylpropyloxy, and the like, which may be substituted.

The term “heterocyclyl” or “heterocyclic” used herein, either alone or in combination with other radicals, denotes saturated, partially saturated and unsaturated ring-shaped radicals, the heteroatoms selected from nitrogen, sulfur and oxygen. Examples of saturated heterocyclic radicals include but not limited to aziridinyl, azetidinyl, pyrrolidinyl, imidazolidinyl, piperidinyl, piperazinyl, 2-oxopiperidinyl, 4-oxopiperidinyl, 2-oxopiperazinyl, 3-oxopiperazinyl, morpholinyl, thiomorpholinyl, 2-oxomorpholinyl, azepinyl, diazepinyl, oxapinyl, thiazepinyl, oxazolidinyl, thiazolidinyl, and the like; examples of partially saturated heterocyclic radicals include dihydrothiophene, dihydropyran, dihydrofuran, dihydrothiazole, and the like.

The term “heteroaryl” or “heteroaromatic” used herein, either alone or in combination with other radicals, denotes unsaturated 5 to 6 membered heterocyclic radicals containing one or more hetero atoms selected from O, N or S, attached to an aryl group, such as pyridyl, thienyl, furyl, pyrrolyl, oxazolyl, thiazolyl, imidazolyl, isoxazolyl, oxadiazolyl, tetrazolyl, benzopyranyl, benzofuranyl, benzothienyl, indolinyl, indolyl, quinolinyl, pyrimidinyl, pyrazolyl, quinazolinyl, pyrimidonyl, benzoxazinyl, benzoxazinonyl, benzothiazinyl, benzothiazinonyl, benzoxazolyl, benzothizaolyl, benzimidazolyl, and the like.

The term “heterocyclylalkyl” used herein, either alone or in combination with other radicals, represents a heterocyclyl group, as defined above, substituted with an alkyl group of one to twelve carbons, such as pyrrolidinealkyl, piperidinealkyl, morpholinealkyl, thiomorpholinealxyl, oxazolinealkyl, and the like, which may be substituted. The term “heteroaralkyl” used herein, either alone or in combination with other radicals, denotes a heteroaryl group, as defined above, attached to a straight or branched saturated carbon chain containing 1 to 6 carbons, such as (2-furyl)methyl, (3-furyl)methyl, (2-thienyl)methyl, (3-thienyl)methyl, (2-pyridyl)methyl, 1-methyl-1-(2-pyrimidyl)ethyl and the like. The terms “heteroaryloxy”, “heteroaralkoxy”, “heterocycloxy”, “heterocylylalkoxy” denotes heteroaryl, heteroarylalkyl, heterocyclyl, heterocyclylalkyl groups respectively, as defined above, attached to an oxygen atom.

The term “acyl” used herein, either alone or in combination with other radicals, denotes a radical containing one to eight carbons such as formyl, acetyl, propanoyl, butanoyl, iso-butanoyl, pentanoyl, hexanoyl, heptanoyl, benzoyl and the like, which may be substituted.

The term “acyloxy” used herein, either alone or in combination with other radicals, denotes a radical acyl, as defined above, directly attached to an oxygen atom, such as acetyloxy, propionyloxy, butanoyloxy, iso-butanoyloxy, benzoyloxy and the like.

The term “acylamino” used herein, either alone or in combination with other radicals, denotes an acyl group as defined earlier, attached to amino group which may be substituted, such as CH₃CONH, C₂H₅CONH, C₃H₇CONH, C₄H₉CONH, C₆H₅CONH and the like, which may be substituted.

The term “mono-substituted amino” used herein, either alone or in combination with other radicals, denotes an amino group, substituted with one group selected from (C₁-C₆)alkyl, substituted alkyl, aryl, substituted aryl or arylalkyl groups. Examples of monoalkylamino group include methylamine, ethylamine, n-propylamine, n-butylamine, n-pentylamine and the like.

The term “disubstituted amino” used herein, either alone or in combination with other radicals, denotes an amino group, substituted with two radicals that may be same or different selected from (C₁-C₆)alkyl, substituted alkyl, aryl, substituted aryl, or arylalkyl groups, such as dimethylamino, methylethylamino, diethylamino, phenylmethyl amino and the like.

The term “arylamino” used herein, either alone or in combination with other radicals, denotes an aryl group, as defined above, linked through amino having a free valence bond from the nitrogen atom, such as phenylamino, naphthylamino, N-methyl anilino and the like.

The term “aralkylamino” used herein, either alone or in combination with other radicals, denotes an arylalkyl group as defined above linked through amino having a free valence bond from the nitrogen atom e.g. benzylamino, phenethylamino, 3-phenylpropylamino, 1-napthylmethylamino, 2-(1-napthyl)ethylamino and the like.

The term “oxo” or “carbonyl” used herein, either alone (—C═O—) or in combination with other radicals, such as “alkylcarbonyl”, denotes a carbonyl radical (—C═O—) substituted with an alkyl radical such as acyl or alkanoyl, as described above.

The term “carboxylic acid” used herein, alone or in combination with other radicals, denotes a —COOH group, and includes derivatives of carboxylic acid such as esters and amides. The term “ester” used herein, alone or in combination with other radicals, denotes —COO— group, and includes carboxylic acid derivatives, where the ester moieties are alkoxycarbonyl, such as methoxycarbonyl, ethoxycarbonyl, and the like, which may be substituted; aryloxycarbonyl group such as phenoxycarbonyl, napthyloxycarbonyl, and the like, which may be substituted; aralkoxycarbonyl group such as benzyloxycarbonyl, phenethyloxycarbonyl, napthylmethoxycarbonyl, and the like, which may be substituted; heteroaryloxycarbonyl, heteroaralkoxycarbonyl, wherein the heteroaryl group, is as defined above, which may be substituted; heterocyclyloxycarbonyl, where the heterocyclic group, as defined earlier, which may be substituted.

The term “amide” used herein, alone or in combination with other radicals, represents an aminocarbonyl radical (H₂N—C═O—), wherein the amino group is mono- or di-substituted or unsubstituted, such as methylamide, dimethylamide, ethylamide, diethylamide, and the like. The term “aminocarbonyl” used herein, either alone or in combination with other radicals, with other terms such as “aminocarbonylalkyl”, “n-alkylaminocarbonyl”, “N-arylaminocarbonyl”, “N,N-dialkylaminocarbonyl”, “N-alkyl-N-arylaminocarbonyl”, “N-alkyl-N-hydroxyaminocarbonyl”, and “N-alkyl-N-hydroxyaminocarbonylalkyl”, substituted or unsubstituted. The terms “N-alkylaminocabonyl” and “N,N-dialkylaminocarbonyl” denotes aminocarbonyl radicals, as defined above, which have been substituted with one alkyl radical and with two alkyl radicals, respectively. Preferred are “lower alkylaminocarbonyl” having lower alkyl radicals as described above attached to aminocarbonyl radical. The terms “N-arylaminocarbonyl” and “N-alkyl-N-arylaminocarbonyl” denote amiocarbonyl radicals substituted, respectively, with one aryl radical, or one alkyl, and one aryl radical. The term “aminocarbonylalkyl” includes alkyl radicals substituted with aminocarbonyl radicals.

The term “hydroxyalkyl” used herein, either alone or in combination with other radicals, denotes an alkyl group, as defined above, substituted with one or more hydroxy radicals, such as hydroxymethyl, hydroxyethyl, hydroxypropyl, hydroxybutyl, hydroxypentyl, hydroxyhexyl and the like.

The term “aminoalkyl” used herein, alone or in combination with other radicals, denotes an amino (—NH₂) moiety attached to an alkyl radical, as defined above, which may be substituted, such as mono- and di-substituted aminoalkyl. The term “alkylamino” used herein, alone or in combination with other radicals, denotes an alkyl radical, as defined above, attached to an amino group, which may be substituted, such as mono- and di-substituted alkylamino.

The term “alkoxyalkyl” used herein, alone or in combination with other radicals, denotes an alkoxy group, as defined above, attached to an alkyl group, such as methoxymethyl, ethoxymethyl, methoxyethyl, ethoxyethyl and the like. The term “aryloxyalkyl” used herein, alone or in combination with other radicals, includes phenoxymethyl, napthyloxymethyl, and the like. The term “aralkoxyalkyl” used herein, alone or in combination with other radicals, includes C₆H₅CH₂OCH₂, C₆H₅CH₂OCH₂CH₂, and the like.

The term “alkylthio” used herein, either alone or in combination with other radicals, denotes a straight or branched or cyclic monovalent substituent comprising an alkyl group of one to twelve carbon atoms, as defined above, linked through a divalent sulfur atom having a free valence bond from the sulfur atom, such as methylthio, ethylthio, propylthio, butylthio, pentylthio and the like. Examples of cyclic alkylthio are cyclopropylthio, cyclobutylthio, cyclopentylthio, cyclohexylthio and the like, which may be substituted.

The term “thioalkyl” used herein, either alone or in combination with other radicals, denotes an alkyl group, as defined above, attached to a group of formula —SR′, where R′ represents hydrogen, alkyl or aryl group, e.g. thiomethyl, methylthiomethyl, phenylthiomethyl and the like, which may be substituted.

The term “arylthio” used herein, either alone or in combination with other radicals, denotes an aryl group, as defined above, linked through a divalent sulfur atom, having a free valence bond from the sulfur atom such as phenylthio, napthylthio and the like.

The term “alkoxycarbonylamino” used herein, alone or in combination with other radicals, denotes an alkoxycarbonyl group, as defined above, attached to an amino group, such as methoxycarbonylamino, ethoxycarbonylamino, and the like. The term “aryloxycarbonylamino” used herein, alone or in combination with other radicals, denotes an aryloxycarbonyl group, as defined above, attached to the an amino group, such as C₆H₅OCONH, C₆H₅OCONCH₃, C₆H₅OCONC₂H₅, C₆H₄(CH₃O)CONH, C₆H₄(OCH₃)OCONH, and the like. The term “aralkoxycarbonylamino” used herein, alone or in combination with other radicals, denotes an aralkoxycarbonyl group, as defined above, attached to an amino group C₆H₅CH₂OCON₁, C₆H₅CH₂CH₂CH₂OCONH, C₆H₅CH₂OCONHCH₃, C₆H₅CH₂OCONC₂H₅, C₆H₄(CH₃)CH₂OCONH, C₆H₄(OCH₃)CH₂OCONH, and the like.

The term “aminocarbonylamino”, “alkylaminocarbonylamino”, “dialkylaminocarbonylamino” used herein, alone or in combination with other radicals, denotes a carbonylamino (—CONH₂) group, attached to amino(NH₂), alkylamino group or dialkylamino group respectively, where alkyl group is as defined above.

The term “amidino” used herein, either alone or in combination with other radicals, denotes a —C(═NH)—NH₂ radical. The term “alkylamidino” denotes an alkyl radical, as discussed above, attached to an amidino group.

The term “guanidino” used herein, either alone or in combination with other radicals, denotes HN═C(NH₂)NH—, suitably substituted with other radicals, such as alkylguanidino, dialkylguanidino, where the alkyl group, as defined above is attached to a guanidino group, such as methylguanidino, ethylguanidino, dimethylguanidino, and the like.

The tem “hydrazino” used herein, either alone or in combination with other radicals, denotes —NHH—, suitably substituted with other radicals, such as alkyl hydrazino, where an alkyl group, as defined above is attached to a hydrazino group.

The term “alkoxyamino” used herein, alone or in combination with other radicals, denotes an alkoxy group, as defined above, attached to an amino group. The term “hydroxyamino” used herein, alone or in combination with other radicals, denotes —NHOH moiety, and may be substituted.

The term “sulfenyl” or “sulfenyl and its derivatives” used herein, alone or in combination with other radicals, denotes a bivalent group, —SO— or RSO, where R is substituted or unsubstituted alkyl, aryl, heteroaryl, heterocyclyl, and the like.

The term “sulfonyl” or “sulfones and its derivatives” used herein, either alone or in combination with other radicals, with other terms such as alkylsulfonyl, denotes divalent radical —SO₂—, or RSO₂—, where R is substituted or unsubstituted groups selected from alkyl, aryl, heteroaryl heterocyclyl, and the like. “Alkylsulfonyl” denotes alkyl radicals, as defined above, attached to a sulfonyl radical, such as methylsulfonyl, ethylsulfonyl, propylsulfonyl and the like. The term “arylsulfonyl” used herein, either alone or in combination with other radicals, denotes aryl radicals, as defined above, attached to a sulfonyl radical, such as phenylsulfonyl and the like.

The term “sulfonic acid or its derivatives”, used herein, either alone or in combination with other radicals, denotes —SO₃H group and its derivatives such as sulfonylamino(SO₂NH₂); N-alkylaminosulfonyl and N,N-dialkylaminosulfonyl radicals where the sulfonylamino group is substituted with one and two alkyl groups respectively, such as N-methylaminosulfonyl, N-ethylaminosulfonyl, N,N-dimethylaminosulfonyl, N-methyl-N-ethylaminosulfonyl and the like; N-arylaminosulfonyl and N-alkyl-N-arylaminosulfonyl groups where the sulfonylamino group is substituted with one aryl radical, or one alkyl and one aryl radical; —SO₃R, wherein ‘R’ represents alkyl, aryl, aralkyl groups, as defined above, which may be substituted.

The term “phosphonic acid or its derivatives”, used herein, either alone or in combination with other radicals, denotes P(O)(OH)₂, P(O)(O(C₁-C₆) alkyl)₂, P(O)(O aryl)₂, P(O)(OH)(O(C₁-C₆)alkyl), and the like.

Suitable groups and substituents on the groups may be selected from those described anywhere in the specification.

Particularly useful compounds of the present invention are:
1-Cyclopropyl-6-fluoro-8-methoxy-7-(2-nitro-6,7-dihydro-4H-thieno[3,2-c]pyridin-5-yl)-4-oxo-1,4-dihydro-quinoline-3-carboxy fluoroborate and its pharmaceutically acceptable salts;
1-Cyclopropyl-6-fluoro-8-methoxy-7-(2-nitro-6,7-dihydro-4H-thieno[3,2-c]pyridin-5-yl)-4-oxo-1,4-dihydro-quinoline-3-carboxylic acid and its pharmaceutically acceptable salts;
1-Cyclopropyl-7-(6,7-dihydro-4H-thieno[3,2-c]pyridin-5-yl)-6-fluoro-4-oxo-1,4-dihydro-quinoline-3-carboxy fluoroborate and its pharmaceutically acceptable salts;
1-Cyclopropyl-7-(6,7-dihydro-4H-thieno[3,2-c]pyridin-5-yl)-6-fluoro-8-methoxy-4-oxo-1,4-dihydro-quinoline-3-carboxy fluoroborate and its pharmaceutically acceptable salts;
1-Cyclopropyl-7-(6,7-dihydro-4H-thieno[3,2-c]pyridin-5-yl)-5,6,8-fluoro-4-oxo-1,4-dihydro-quinoline-3-carboxy fluoroborate and its pharmaceutically acceptable salts;
7-(2-bromo-6,7-dihydro-4H-thieno[3,2-c]pyridin-5-yl)-1-cyclopropyl-8-methoxy-4-oxo-1,4-dihydro-quinoline-3-carboxy fluoroborate and its pharmaceutically acceptable salts;
1-Cyclopropyl-7-(6,7-dihydro-4H-thieno[3,2-c]pyridin-5-yl)-6-fluoro-4-oxo-1,4-dihydro-quinoline-3-carboxylic acid and its pharmaceutically acceptable salts;
1-Cyclopropyl-7-(6,7-dihydro-4H-thieno[3,2-c]pyridin-5-yl)-6-fluoro-8-methoxy-4-oxo-1,4-dihydro-quinoline-3-carboxylic acid and its pharmaceutically acceptable salts;
1-Cyclopropyl-7-(6,7-dihydro-4H-thieno[3,2-c]pyridin-5-yl)-6-fluoro-8-methoxy-5-nitro-4-oxo-1,4-dihydro-quinoline-3-carboxylic acid and its pharmaceutically acceptable salts;
5-Acetylamino-1-cyclopropyl-7-(6,7-dihydro-4H-thieno[3,2-c]pyridin-5-yl)-6,8-difluoro-4-oxo-1,4-dihydro-quinoline-3-carboxylic acid and its pharmaceutically acceptable salts;
5-Amino-1-cyclopropyl-7-(6,7-dihydro-4H-thieno[3,2-c]pyridin-5-yl)-6,8-difluoro-4-oxo-1,4-dihydro-quinoline-3-carboxylic acid and its pharmaceutically acceptable salts;
1-Cyclopropyl-7-(6,7-dihydro-4H-thieno[3,2-c]pyridin-5-yl)-5,6,8-fluoro-4-oxo-1,4-dihydro-quinoline-3-carboxylic acid and its pharmaceutically acceptable salts;
7-(2-bromo-6,7-dihydro-4H-thieno[3,2-c]pyridin-5-yl)-1-cyclopropyl-6-fluoro-8-methoxy-4-oxo-1,4-dihydro-quinoline-3-carboxylic acid and its pharmaceutically acceptable salts;
7-(2-bromo-6,7-dihydro-4H-thieno[3,2-c]pyridin-5-yl)-1-cyclopropyl-6-fluoro-4-oxo-1,4-hydro-quinoline-3-carboxylic acid and its pharmaceutically acceptable salts;
5-Amino-1-cyclopropyl-7-(2-bromo-6,7-dihydro-4H-thieno[3,2-c]pyridin-5-yl)-6,8-difluoro-4-oxo-1,4-dihydro-quinoline-3-carboxylic acid and its pharmaceutically acceptable salts;
7-(2-carboxy-6,7-dihydro-4H-thieno[3,2-c]pyridin-5-yl)-1-cyclopropyl-6-fluoro-8-methoxy-4-oxo-1,4-dihydro-quinoline-3-carboxylic acid and its pharmaceutically acceptable salts;
1-Cyclopropyl-6-fluoro-7-(2-formyl-6,7-dihydro-4H-thieno[3,2-c]pyridin-5-yl)-8-methoxy-4-oxo-1,4-dihydro-quinoline-3-carboxylic acid and its pharmaceutically acceptable salts;
1-Cyclopropyl-6-fluoro-8-methoxy-7-(4-methyl-6,7-dihydro-4H-fluoro[3,2-c]pyridin-5-yl)-4-oxo-1,4-dihydro-quinoline-3-carboxylic acid and its pharmaceutically acceptable salts;
1-Cyclopropyl-6-fluoro-7-(2-acetoxy-6,7-dihydro-4H-thieno[3,2-c]pyridin-5-yl)-8-methoxy-4-oxo-1,4-dihydro-quinoline-3-carboxylic acid and its pharmaceutically acceptable salts;
1-Cyclopropyl-6-fluoro-7-(2-hydroxyimino-6,7-dihydro-4H-thieno[3,2-c]pyridin-5-yl)-8-methoxy-4-oxo-1,4-dihydro-quinoline-3-carboxylic acid and its pharmaceutically acceptable salts;
1-Cyclopropyl-7-(2-formyl-6,7-dihydro-4H-thieno[3,2-c]pyridin-5-yl)-5,6,8-trifluoro-4-oxo-1,4-dihydro-quinoline-3-carboxylic acid and its pharmaceutically acceptable salts;
1-Cyclopropyl-6-fluoro-8-methoxy-7-(7-methyl-6,7-dihydro-4H-thieno[3,2-c]pyridin-5-yl)-4-oxo-1,4-dihydro-quinoline-3-carboxylic acid and its pharmaceutically acceptable salts;
1-Cyclopropyl-6-fluoro-7-(2-hydroxymethyl-6,7-dihydro-4H-thieno[3,2-c]pyridin-5-yl)-8-methoxy-4-oxo-1,4-dihydro-quinoline-3-carboxylic acid and its pharmaceutically acceptable salts;
1-Cyclopropyl-7-(2-formyl-6,7-dihydro-4H-thieno[3,2-c]pyridin-5-yl)-5,6,8-trifluoro-4-oxo-1,4-dihydro-quinoline-3-carboxy fluoroborate and its pharmaceutically acceptable salts;
1-Cyclopropyl-7-(2-nitro-6,7-dihydro-4H-thieno[3,2-c]pyridin-5-yl)-5,6,8-trifluoro-4-oxo-1,4-dihydro-quinoline-3-carboxy fluoroborate and its pharmaceutically acceptable salts;
1-Cyclopropyl-7-(2-nitro-6,7-dihydro-4H-thieno[3,2-c]pyridin-5-yl)-5,6,8-trifluoro-4-oxo-1,4-dihydro-quinoline-3-carboxylic acid and its pharmaceutically acceptable salts;

The compounds of formula (I), where all the substituents are as described elsewhere in the specification may be synthesized using the methods described below, together with conventional techniques known to those skilled in the art of organic synthesis, or variations thereon as appreciated by those skilled in the art. Referred methods include, but not limited to those described below.

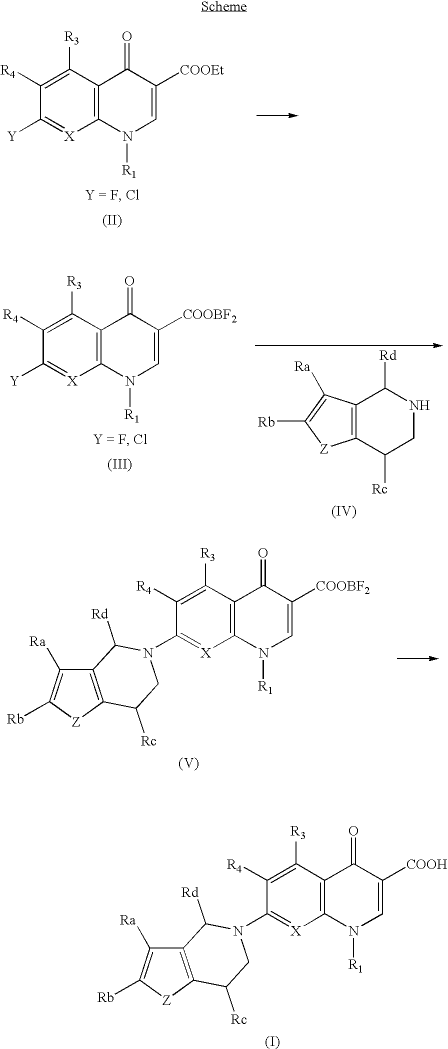

Compound of general formula (II) can be converted to their corresponding fluoborate ester of formula (III) through trans esterification using fluoboric acid at a temperature ranging from 40° C. to 100° C. The compound of formula (III) is reacted with compound of general formula (IV) to give compound of general formula (V), using suitable organic bases such as triethylamine, N—N-diisopropyl ethyl amine, ammonia solution and the like in solvents such as DMF, DMSO, pyridine, acetonitrile and the like or their mixtures thereof, at a temperature ranging from 15° C. to 80° C. Compound of formula (V) represents compounds of formula (I) where all symbols are as defined earlier and R₂=—OBF₂. The fluoborate ester of formula (V) can be hydrolyzed to give compound of formula (I), where all symbols are as defined earlier and R₂=OH. Suitable hydrolyzing agent may be selected from alcoholic KOH or NaOH and heating at 60-80° C. or using organic bases such as triethylamine, pyridine, piperidine and the like or their mixture thereof in 80-100% ethanol-water at reflux temperature. The compounds of formula (Ia) may be optionally converted to their corresponding esters, amides, acid salts by processes known.

The pharmaceutically acceptable salts forming a part of this invention can be prepared by treating the compound of formula (I) with 1-6 equivalents of a base such as sodium hydride, sodium methoxide, sodium ethoxide, sodium hydroxide, potassium tert-butoxide, calcium hydroxide, calcium acetate, calcium chloride, magnesium hydroxide, magnesium chloride, magnesium alkoxide and the like. Solvents such as water, acetone, ether, THE, methanol, ethanol, t-butanol, 2-butanone, dioxane, propanol, butanol, isopropanol, diisopropyl ether, tert-butyl ether or mixtures thereof may be used. Organic bases such as lysine, arginine, methyl benzylamine, ethanolamine, diethanolamine, tromethamine, choline, guanidine and their derivatives may be used. Acid addition salts, wherever applicable may be prepared by treatment with acids such as tartaric acid, mandelic acid, fumaric acid, malic acid, lactic acid, maleic acid, salicylic acid, citric acid, ascorbic acid, benzene sulfonic acid, p-toluene sulfonic acid, hydroxynaphthoic acid, methane sulfonic acid, acetic acid, benzoic acid, succinic acid, palmitic acid, hydrochloric acid, hydrobromic acid, sulfuric acid, nitric acid and the like in solvents such as water, alcohols, ethers, ethyl acetate, dioxane, THF, acetonitrile, DMF or a lower alkyl ketone such as acetone, or mixtures thereof.

These salts may be in hydrated form—some of the compounds of the invention may form metal salts such as sodium, potassium, calcium and magnesium salts and these are embraced by the term “pharmaceutically acceptable salts”.

It will be appreciated that in any of the above mentioned reactions any reactive group in the substrate molecule may be protected, according to conventional chemical practice. Suitable protecting groups in any of the above mentioned reactions are those used conventionally in the art. The methods of formation and removal of such protecting groups are those conventional methods appropriate to the molecule being protected. T. W. Greene and P. G. M. Wuts “Protective groups in Organic Synthesis”, John Wiley & Sons, Inc, 1999, 3^{rd} Ed., 201-245 along with references therein gives such conventional methods and are incorporated herein as references.

It will be appreciated that the above-mentioned preparation of the compounds of Formula (I), or a pharmaceutically acceptable salts thereof, and/or pharmaceutically acceptable solvates thereof may exist either as a racemate or in optically pure form. Both the racemate and the stereoisomers are encompassed by the present invention.

Another aspect of the present invention comprises a pharmaceutical composition, containing at least one of the compounds of the general formula (I), their tautomeric forms, their stereoisomers, their pharmaceutically acceptable salts, their pharmaceutically acceptable solvates as an active ingredient, together with pharmaceutically employed carriers diluents and the like.

Pharmaceutical compositions containing a compound of the present invention may be prepared by conventional techniques as are well known in the art.

The compounds of Formula (I) are useful in the treatment of microbial infections in humans and other warm blooded animals, by either oral, topical or parenteral or other conventional techniques as are well known. Besides being useful for human treatment, these compounds are also useful for veterinary treatment of companion animals, exotic animals and farm animals including mammals, rodents, and the like. More preferred animals include horses, dogs and cats.

For the treatment of any of the above-mentioned diseases the compounds of formula (I) may be administered, for example, orally, topically, parenterally, in dosage unit formulations containing conventional non-toxic pharmaceutically acceptable carriers, adjuvants and vehicles.

The pharmaceutical composition is provided by employing conventional techniques. Preferably the composition is in unit dosage form containing an effective amount of the active component, that is, the compounds of formula (I) according to this invention.

The quantity of active component, that is, the compounds of formula (I) according to this invention, in the pharmaceutical composition and unit dosage form thereof may be varied or adjusted widely depending upon the particular application method, the potency of the particular compound and the desired concentration. Generally, the quantity of active component will range between 0.5% to 90% by weight of the composition.

In therapeutic use for treating bacterial infections in humans and animals that have been diagnosed with having bacterial infections, the compounds or pharmaceutical compositions thereof will be administered orally, parenterally and/or topically at a dosage to obtain and maintain a concentration, that is, an amount, or blood-level of active component in the animal undergoing treatment which will be antibacterially active. Generally, such antibacterially effective amount of dosage of active component will be in the range of about 0.1 to about 100 mg/kg, more preferably about 0.5 to about 100 mg/kg of body weight/day. However, it should be appreciated that the dosages may vary depending upon the requirements of the patient, the severity of the bacterial infection, and the particular compound being used. Also, it must be understood that the initial dosage administered may be increased beyond the upper level in order to rapidly achieve the desired blood level or the initial dosage may be smaller than the optimum and the and the daily dosage may be progressively increased during the course of treatment depending on the particular situation. If desired, the daily dose may also be divided into multiple doses for administration, e.g. two to four times per day.

The compounds of the present invention may be administered alone or in combination with pharmaceutically acceptable carriers or diluents by any of the routes as previously indicated, in single or multiple doses.

The pharmaceutically acceptable compounds of the present invention are useful antibacterial agents having a good spectrum of activity in vitro and against standard Gram-positive organisms, which are used to screen for activity against pathogenic bacteria. Notably, the pharmaceutically acceptable compounds of the present invention show activity against enterococci pneumococci and methicillin resistant strains of S. aureus and coagulase negative staphylococci together with morganella strains. The antibacterial spectrum and potency of a particular compound may be determined in a standard test system. The activity is described in terms of the minimum inhibitory concentration (MIC) determined by microbroth dilution technique as per NCCLS standards.

The invention is explained in detail by the examples given below, which illustrative and therefore should not be construed to limit the scope of the invention.

1H MR spectral data given in the tables (vide infra) are recorded using a 300 MHz spectrometer (Bruker AVANCE-300) and reported in 8 scale. Until and otherwise mentioned the solvent used for NMR is CDCl₃ using Tetramethyl silane as the internal standard.

Preparation 1

### 1-Cyclopropyl-7-(6,7-dihydro-4H-thieno[3,2-c]pyridin-5-yl)-6-fluoro-8-methoxy-4-oxo-1,4-dihydro-quinoline-3-carboxy fluoroborate. (Compound no. 01)

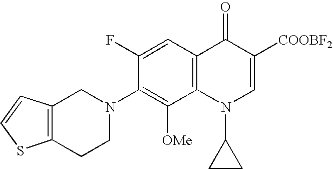

To (0.5 g, 1.4 mM) 1-cyclopropyl-6,7-difluoro-8-methoxy-4-oxo-1,4-dihydro-quinoline-3-carboxy fluoroborate in 10 mL DMSO was added (0.4 g, 2.92 mM) 4,5,6,7-tetrahydro-thieno[3,2-]pyridine in 10 mL DMSO dropwise at 0-5° C. Then stirring was continued at ca. 25° C. for 1 h and completion of reaction was monitored by TLC. Reaction mixture was poured in 100 ml of D.M. water and the solid precipitated was collected on buchner funnel which was then washed with 25 ml of di-isopropyl ether to afford pale yellow solid (0.3 g, 47%). m.p: 214-216° C.

The following compounds were prepared following the above procedure.

TABLE 1

| | |
|---|---|
| | (I) |
| 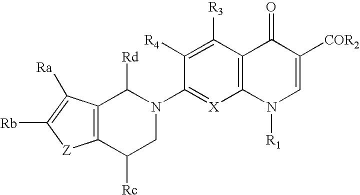 | |

| Sr. | | | | | | | | | | | Mol. | Yield |
|---|---|---|---|---|---|---|---|---|---|---|---|---|
| No. | Z | Ra | Rb | Rc | Rd | R₁ | R₂ | R₃ | R₄ | X | Wt. | (%) |
| 01 | S | H | H | H | H | 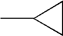 | OBF₂ | H | F | COMe | 462 | 47 |
| | (DMSO-d₆): −9.06(1H, s), 7.93(1H, d, J=12.0Hz), 7.37(1H, d, J=5.40Hz), 6.90(1H, d, | | | | | | | | | | | |
| | J=5.10Hz), 4.53(2H, s), 4.40(1H, m), 3.77(2H, J=4.20Hz), 3.71(3H, s), | | | | | | | | | | | |
| | 3.03(2H, bs), 1.20 (4H, d J=5.60Hz). | | | | | | | | | | | |
| 02 | S | H | H | H | H | 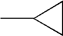 | OBF₂ | H | F | CH | 432 | 68 |
| | (DMSO-d₆): −8.98(1H, s), 8.13(1H,d, J=13.40Hz), 7.73(1H, d, J=7.49Hz), 7.40(1H, d, | | | | | | | | | | | |
| | J=5.40Hz), 6.97(1H, d, J=5.40Hz), 4.65(2H, s), 4.02(1H, m), | | | | | | | | | | | |
| | 3.88(2H, t, J=5.0Hz), 3.03(2H, bs), 1.16(4H, m). | | | | | | | | | | | |
| 03 | S | H | H | H | H | 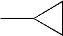 | OBF₂ | NO₂ | F | COMe | 507 | 67 |
| 04 | S | H | H | H | H | 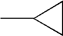 | OBF₂ | F | F | CF | 468 | 59 |
| 05 | S | H | H | H | H | 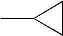 | OBF₂ | NH₂ | F | CF | 465 | |
| 06 | S | H | Br | H | H | 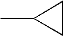 | OBF₂ | H | F | COMe | 541 | 43 |
| | (DMSO-d₆): −9.06(1H, s), 7.96(1H, d, J=12.10Hz), 7.07(1H, s), 4.42(3H, m), | | | | | | | | | | | |
| | 3.70(5H, m), 2.9(2H, s), 1.26(4H, m). | | | | | | | | | | | |
| 07 | S | H | NO₂ | H | H | 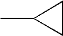 | OBF₂ | H | F | COMe | 507 | 85 |
| 08 | S | H | CHO | H | H | 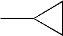 | OBF₂ | H | F | COMe | 490 | 30 |
| 09 | S | H | CH₂OH | H | H | 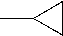 | OBF₂ | H | F | COMe | 492 | 80 |
| 10 | S | H | COOH | H | H | 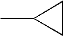 | OBF₂ | H | F | COMe | 506 | |
| 11 | O | H | H | H | CH₃ | 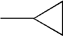 | OBF₂ | H | F | COMe | 460 | 35 |
| 12 | S | H | H | CH₃ | H | 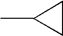 | OBF₂ | H | F | COMe | 476 | |

Preparation 2

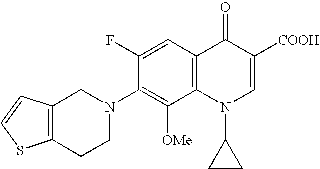

### 1-Cyclopropyl-7-(6,7-dihydro-4H-thieno[3,2-c]pyridin-5-yl)-6-fluoro-8-methoxy-4-oxo-1,4-dihydro-quinoline-3-carboxylic acid. (compound no. 13)

To (0.156 g, 0.337 mM) of 1-cyclopropyl-7-(6,7-dihydro-4H-thieno[3,2-c]pyridin-5-yl)-6-fluoro-8-methoxy-4-oxo-1,4-dihydro-quinoline-3-carboxy fluoroborate (compound 01, obtained above) dissolved in 10 mL 80% aq. ethanol was added 0.23 mL triethylamine dropwise at ca 27° C. The reaction mixture was refluxed for 1 h (TLC). Solvents were removed in vacuum and the concentrate obtained was dissolved in 100 mL of dichloromethane and extracted with 3×50 ml D.M water. Dichloromethane layer was separated and dried over anhydrous Na₂SO₄. The solvent was removed on rotary evaporator to give brown solid which was purified through column chromatography using silica gel as stationary phase and 0-5% methanol in chloroform as eluent. The concentration of required fractions afforded offwhite solid (0.118 g, 85%), m.p 173-175° C.

The following compounds were prepared following the above procedure.

TABLE 2

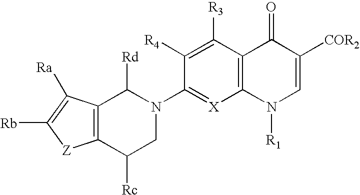

| Sr. | | | | | | | | | | | Mol. | Yield |
|---|---|---|---|---|---|---|---|---|---|---|---|---|
| No. | Z | Ra | Rb | Rc | Rd | R₁ | R₂ | R₃ | R₄ | X | Wt. | (%) |
| 13 | S | H | H | H | H | 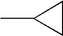 | OH | H | F | COMe | 414 | 85 |
| | (DMSO-d₆): −14.98(1H, s), 8.70(1H, s), 7.77(1H, d, J=12.0Hz), 7.35(1H, d, | | | | | | | | | | | |
| | J=5.10Hz), 6.91(1H, d, J=5.10Hz), 4.42(2H, s), 4.18(1H, m), 3.70(3H, s), 3.64(2H, t, | | | | | | | | | | | |
| | J=4.81Hz), 2.98(2H, bs), 1.04-1.14(4H, m). | | | | | | | | | | | |
| 14 | S | H | H | H | H | 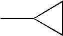 | OH | H | F | CH | 384 | 70 |
| | (DMSO-d₆): −15.26(1H, s), 8.64(1H, s), 7.95(1H, d, J=13.20Hz), 7.61(1H, d, | | | | | | | | | | | |
| | J=7.50Hz,), 7.39(1H, d, J=5.10Hz), 6.97(1H, d, | | | | | | | | | | | |
| | J=5.10Hz), 4.51(2H, s), 3.74(3H, m), 2.98(2H, s) 1.24(4H, m). | | | | | | | | | | | |
| 15 | S | H | H | H | H | 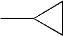 | OH | NO₂ | F | COMe | 459 | 43 |
| | (DMSO-d₆): −13.86(1H, s), 8.74(1H, s), 7.35(1H, d, J=5.10Hz), 6.91(1H, d, | | | | | | | | | | | |
| | J=5.10Hz), 4.48(2H, s), 4.19(1H, m), 3.77(4H, m), 3.71(5H, m), 2.99(2H, s). | | | | | | | | | | | |
| 16 | S | H | H | H | H | 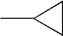 | OH | F | F | CF | 420 | 89 |
| | (DMSO-d₆ + Drops of CD₃OD): −14.74(1H, s), 8.64(1H, s), 7.37(1H, d, J=5.10Hz), | | | | | | | | | | | |
| | 6.89(1H, d, J=5.10Hz), 4.54(2H, s), 4.09(1H, m), 3.71(2H, t, J=5.10Hz),), 3.0(2H, t, | | | | | | | | | | | |
| | J=4.20Hz), 1.14(4H, m). | | | | | | | | | | | |
| 17 | S | H | H | H | H | 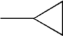 | OH | NH₂ | F | CF | 417 | 78 |
| | (CDCl₃): −14.68(1H, s), 8.66(1H, s), 7.16(1H, d, J=1.54Hz), 6.81(1H, d, J=1.54Hz), | | | | | | | | | | | |
| | 6.51(2H, s), 4.52(2H, s), 3.97(1H, m), 3.68(2H, m), 3.0(2H, m), | | | | | | | | | | | |
| | 1.08(4H, m). | | | | | | | | | | | |
| 18 | S | H | Br | H | H | 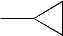 | OH | H | F | COMe | 493 | 60 |
| | (DMSO-d₆): −8.70(1H, s), 7.78(1H, d, J=12.11Hz), 7.05(1H, s), 4.36(2H, s), | | | | | | | | | | | |
| | 4.16(1H, m), 3.70(3H, s), 3.0(4H, s), 2.91(2H, s), 1.25(1H, | | | | | | | | | | | |
| | t, J=7.0Hz). | | | | | | | | | | | |
| 19 | S | H | NO₂ | H | H | 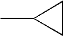 | OH | H | F | COMe | 459 | 60 |
| | (CDCl₃): −14.64(1H, s), 8.85(1H, s), 7.93(1H, d, J=12.0Hz), 7.71(1H, s), | | | | | | | | | | | |
| | 4.45(2H, s), 4.05(1H, m), 3.77(3H, s), 3.74(2H, t, J=1.98Hz) 3.12(2H, t, J=5.10Hz), | | | | | | | | | | | |
| | 1.27(4H, m). | | | | | | | | | | | |
| 20 | S | H | CHO | H | H | 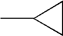 | OH | H | F | COMe | 442 | 28 |
| | (DMSO-d₆): −14.93(1H, s), 9.85(1H, s), 8.71(1H, s), 7.85(1H, s), 7.77(1H, d, | | | | | | | | | | | |
| | J=12.30Hz), 4.48(2H, s), 4.17(1H, m), 3.71(3H, s), 3.38(2H, t, J=5.25Hz),), | | | | | | | | | | | |
| | 3.11(2H, t, J=5.10Hz), 1.10(4H, m). | | | | | | | | | | | |
| 21 | S | H | CH₂OH | H | H | 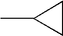 | OH | H | F | COMe | 444 | 18 |
| | (DMSO-d₆ + Drops of CD₃OD): −8.83(1H, s), 7.90(1H, d, J=12.01Hz), 6.73(1H, s), | | | | | | | | | | | |
| | 5.32(1H, s), 4.74(2H, s), 4.42(2H, s), 4.08(1H, m), 3.73(5H, m), 3.04(2H, bs), | | | | | | | | | | | |
| | 1.27(3H, m), 1.05(2H, bs). | | | | | | | | | | | |
| 22 | S | H | COOH | H | H | 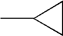 | OH | H | F | COMe | 458 | 36 |
| | (DMSO-d₆ + Drops of CD₃OD): −8.84(1H, s), 7.93(1H, d, J=12.09Hz), 7.54(1H, s), | | | | | | | | | | | |
| | 4.47(2H, s), 4.15(1H, m), 3.74(5H, m), 3.40(4H, m), 1.25(2H, s), 1.24(2H, s). | | | | | | | | | | | |
| 23 | O | H | H | H | CH₃ | 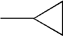 | OH | H | F | COMe | 412 | 20 |
| | (CDCl₃): −14.77(1H, s), 8.88(1H, s), 7.95(1H, d, J=1.56Hz), 7.34(1H, s), 6.30(1H, d, | | | | | | | | | | | |
| | J=1.56Hz), 4.73(1H, m), 4.15(1H, m), 4.01(1H, m), 3.83(3H, s), 3.70(1H, m), | | | | | | | | | | | |
| | 3.55(1H, m), 1.58(3H, s), 1.32(2H, m), 1.15(2H, m), 0.93(1H, m) | | | | | | | | | | | |
| 24 | S | H | COONa | H | H | 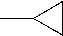 | OH | H | F | COMe | 480 | 76 |
| | (CDCl₃ + Drops of CD₃OD): −7.93(1H, d, J=12.09Hz), 7.54(1H, s), 4.47(2H, s), | | | | | | | | | | | |
| | 4.15(1H, m), 3.74(5H, m), 3.40(4H, m), 1.25(2H, s), 1.24(2H, s). | | | | | | | | | | | |
| 25 | S | H | CHNOH | H | H | 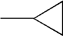 | OH | H | F | COMe | 457 | 65 |
| | (DMSO-d₆) 14.89(1H, s), 11.76(1H, s), 8.70(1H, s), 7.74(2H, m), 7.24(1H, s) | | | | | | | | | | | |
| | 4.17(2H, d, J=3.99Hz), 3.65(8H, d, J=14.0Hz), 1.05(4H, t, J=7.65). | | | | | | | | | | | |
| 26 | S | H | CH₂OAc | H | H | 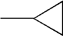 | OH | H | F | COMe | 486 | 4 |

(CDCl₃): −14.30(1H, bs), 8.82(1H, s), 7.92(1H, t, J=9.0Hz), 6.81(1H, s),
5.21(2H, m), 4.40(2H, s), 4.03(1H, bs), 3.69(5H, m), 3.03(2H, d, J=5.31Hz),
2.09(3H, s), 1.23(2H, d, J=6.75Hz), 1.02(2H, m).

Determination of Antibacterial Activity:

The minimum inhibitory concentrations (MIC's) of the compounds for the microorganisms listed in Table A were determined by preparing working solution for each compound of concentration of 128 μg/ml after dissolving it in DMSO. Two-fold serial dilution of the above solution was prepared in duplicates, using Mueller Hinton Broth, in 96 well Tissue culture plate with cover flat bottom wells to give a final volume of 150 μg/ml and concentration of compound ranging from 64 μg/ml-0.12 μg/ml. 30 μg/ml of Standard suspension of each organism which was prepared with turbidity equivalent to the 1:10 diluted 0.5 McFarland standard with density 10⁷ CFU/ml, was added to each well to get approximately a density of 10⁵ CFU/ml. These 96-well Tissue culture plate containing the test samples and positive and negative controls, were incubated at 37° C. for 16-18 hrs. The wells were visually inspected for growth and were also read at 630 nm by Automated Microplate Reader [(EL800) Trinity Biotech.] and the MIC's were recorded as the lowest concentration of drug which inhibits the growth of bacteria. The compounds inhibited the growth of these bacteria with MIC's in a range of about 0.25 μg/ml to about 64 μg/ml.

Results:

Guide to table abbreviations:

Bp: Bacillus pumilus MTCC 1607
S.e.: Staphylococcus epidermidis MTCC 155
S.p.: Staphylococcus pyogenes MTCC 442
S.a.1: Staphylococcus aureus MTCC 96
S.a.2: Staphylococcus aureus ATCC 14154
S.a.3: Staphylococcus aureus ATCC 25923
S.a.4: Staphylococcus aureus ATCC 29213
E.f.1: Enterococcus faecalis MTCC 439
E.f.2: Enterococcus faecalis ATCC 14506
K.0p.: Klebsiella pneumoniae ATCC 10031

P.a.: Pseudomonas aeruginosa ATCC 27853

TABLE
(μg/ml) in vitro activity:

| Compd. No. | Bp | Se | Sp | Sa1 | Sa2 | Sa3 | Sa4 | Ef1 | Ef2 | Kp | Pa |
|---|---|---|---|---|---|---|---|---|---|---|---|
| 13 | ≦0.12 | ≦0.12 | ≦0.12 | ≦0.12 | ≦0.12 | 0.5 | 0.25 | ≦0.12 | 0.25 | 4 | 16 |
| 14 | ≦0.12 | 0.25 | ND | 0.25 | 0.25 | 0.5 | 0.5 | ND | ND | >64 | ND |
| 15 | 0.25 | ND | ND | 1 | ND | ND | ND | ND | ND | >64 | ND |
| 16 | 0.25 | ND | ND | 0.25 | ND | ND | ND | ND | ND | ND | >64 |
| 17 | ≦0.12 | ≦0.12 | ≦0.12 | ≦0.12 | ≦0.12 | 0.5 | 0.25 | ≦0.12 | 0.25 | 4 | 16 |
| 19 | ≦0.12 | ≦0.12 | ≦0.12 | 0.25 | ND | ND | ND | ≦0.12 | 0.25 | 0.25 | 1 |
| 20 | ≦0.12 | 0.25 | 0.25 | ≦0.12 | ND | ND | ND | ≦0.12 | ND | 0.25 | 8 |
| 21 | ≦0.12 | 0.25 | ND | 0.25 | ND | ND | ND | 0.25 | ND | ND | 16 |
| 22 | 0.5 | ND | ND | 4 | ND | ND | ND | 8 | ND | 2 | >16 |
| 23 | ≦0.12 | ND | ND | 8 | ND | ND | ND | 2 | ND | 0.5 | >16 |
| Cipro | <0.12 | 0.25 | 0.5 | 0.25 | 0.5 | 0.5 | 0.25 | 0.25 | 0.25 | 0.25 | 0.5 |
| floxacin | | | | | | | | | | | |
| Gati | ≦0.12 | ≦0.12 | 0.5 | ≦0.12 | ≦0.12 | 0.25 | 0.12 | 0.5 | 0.5 | 0.5 | 4 |
| floxacin | | | | | | | | | | | |

1. A compound of formula (I), their stereoisomers, tautomeric forms, their pharmaceutically acceptable salts, their pharmaceutically acceptable solvates, and pharmaceutical compositions containing them. 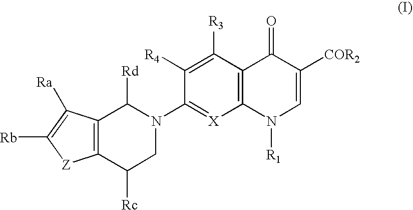
wherein
R₁ represents hydrogen, linear or branched, substituted or unsubstituted groups selected from (C₁-C₁₂) alkyl, (C₂-C₁₂) alkenyl, (C₂-C₁₂) alkynyl, (C₃-C₁₂) cycloalkyl; substituted or unsubstituted groups selected from aryl, heteroaryl or heterocyclic groups; R₂ is selected from hydrogen, —OBF₂ or —OR₆,
Where R₆ represents hydrogen, (C₁-C₆) alkyl, (C₃-C₆)alkenyl or (C₃-C₆)alkynyl groups, which may optionally be substituted; R₃ represents H, OH, linear or branched, substituted or unsubstituted groups selected from —O(C₁-C₁₂) alkyl, —O(C₂-C₁₂)alkenyl, —O(C₂-C₁₂) alkynyl, halo, NO₂, CN, or NR′R″ groups, where R′R″ may be same or different and independently represent H, linear or branched, substituted or unsubstituted groups selected from (C₁-C₆) alkyl, (C₂-C₆ alkynyl or acyl groups; R₄ represents H or halogen atom; X represents N or C—R₇, where R₇ represents H, —OH, —(Oₙ(C₁-C₆) substituted or unsubstituted alkyl where n is 0 or 1, —NO₂, —NH₂, —NHCOCH₃, —CN, —COOH groups; R₁ and R₇ can be taken together with the atoms to which they are attached to form a cyclic ring, which may optionally be substituted and may also optionally contain from 1 to 3 heteroatoms selected from O, N and S;
Ra, Rb may be same or different and represents hydrogen, halogen, haloalkyl, perhaloalkyl, haloalkoxy, perhaloalkoxy, hydroxy, thio, amino, nitro, cyano, formyl, or substituted or unsubstituted groups selected from linear or branched (C₁-C₁₂) alkyl, linear or branched (C₁-C₁₂) alkenyl, linear or branched (C₁-C₁₂) alkynyl, (C₃-C₇) cycloalkyl, (C₃-C₇) cycloalkenyl, bicycloalkyl, bicycloalkenyl, (C₁-C₁₂) alkoxy, (C₁-C₁₂) alkenoxy, cyclo(C₃-C₇)alkoxy, aryl, aryloxy, aralkyl, ar(C₁-C₁₂)alkoxy, heterocyclyl, heteroaryl, heterocyclyl(C₁-C₁₂)alkyl, heteroar (C₁-C₁₂) alkyl, heteroaryloxy, heteroar(C₁-C₁₂)alkoxy, heterocycloxy, heterocyclylalkyloxy, acyl, acyloxy, acylamino, mono-substituted or di-substituted amino, arylamino, aralkylamino, carboxylic acid and its esters and amides, hydroxyalkyl, aminoalkyl, mono-substituted or di-substituted aminoalkyl, alkoxyalky, aryloxyalkyl, aralkoxyalkyl, (C₁-C₁₂)alkylthio, thio(C-C₁₂)alkyl, arylthio, (C₁-C₁₂)alkoxycarbonylamino, aryloxycarbonylamino, aralkyloxycarbonylamino, aminocarboylamino, alkylaminocarbonylamino, alkylamidino, alkylguanidino, dialkylguanidino, hydrazine, alkyl hydrazine, alkoxyamino, hydroxylamino, sufenyl and sulfonyl groups, sulfonic acid, phosphonic acid; R_{c} & R_{d} may be same or different and represents hydrogen, substituted or unsubstituted groups selected from alkyl, alkenyl groups; Z represents O, S or NH, which may optionally be substituted;

2. A compound as claimed in claim 1 wherein the substituents on R₁, R₂, R₃, R₆, R₇, R′, R″, X, Ra, Rb, Rc & Rd are selected from hydroxyl, oxo, halo, thio, nitro, amino, cyano, formyl, amidino, guanidine, hydrazino, alkyl, haloalkyl, perthaoloalkyl, alkoxy, haloalkoxy, perhaloalkoxy, alkenyl, alkynyl, cycloalkyl, cycloalkenyl, bicycloalkyl, bicycloalkenyl, alkoxy, alkenoxy, cycloalkoxy, aryl, aryloxy, aralkyl, aralkoxy, heterocylyl, heteroaryl, heterocyclyalkyl, heteroaralkyl, heteroaryloxy, heteroaralkoxy, heterocyclyloxy, heterocyclylalkoxy, heterocyclylalkoxyacyl, acyl, acyloxy, acylamino, monosubstituted or disubstituted amino, arylamino, aralkylamino, carboxylic acid and its derivatives such as esters and amides, carbonylamino, hydroxyalkyl, aminoalkyl, alkoxyalkyl, aryloxyalkyl, aralkoxyalkyl, alkylthio, thioalkyl, arylthio, alkoxycarbonylamino, aryloxycarbonylamino, aralkyloxycarbonylamino, aminocarbonylamino, alkylaminocarbonylamino, alkoxyamino, hydroxylamino, sulfenyl derivatives, sulfonyl derivatives, sulfonic acid and its derivatives, phosphonic acid and its derivatives.

3. A compound as claimed in claim 1 wherein R₂ represents —OBF₂ or —OH group.

4. A compound according to claim 1 which is selected from:
1-Cyclopropyl-6-fluoro-8-methoxy-7-(2-nitro-6,7-dihydro-4H-thieno[3,2-c]pyridine-5-yl)-4-oxo-1,4-dihydro-quinoline-3-carboxy fluoroborate and its pharmaceutically acceptable salts; 1-Cyclopropyl-6-fluoro-8-methoxy-7-(2-nitro-6,7-dihydro-4H-thieno[3.2-c]pyridine-5-yl)-4-oxo-1,4-dihydro-quinoline-3-carboxylic acid and its pharmaceutically acceptable salts; 1-Cyclopropyl-7-(6,7-dihydro-4H-thieno[3,2-c]pyridine-5-yl)-6-fluoro-4-oxo-1,4-dihydro-quinoline-3-carboxy fluoroborate and its pharmaceutically acceptable salts;
1-Cyclopropyl-7-(6,7-dihydro4H-thieno[3,2-c]pyridine-5-yl)-6-fluoro-8 methoxy-4-oxo-1,4-dihydro-quinoline-3-carboxy fluoroborate and its pharmaceutically acceptable salts;
1-Cyclopropyl-7-(6,7-dihydro-4H-thieno[3,2-c]pyridine-5-yl)-5,6,8-trifluoro-4-oxo-1,4-dihydro-quinoline-3-carboxy fluoroborate and its pharmaceutically acceptable salts;
1-Cyclopropyl-6-fluoro-8-methoxy-7-(7-methyl-6,7-dihydro-4H-thieno[3,2-c]pyridine-5-yl)-4-oxo-1,4-dihydro-quinoline-3-carboxylic acid and its pharmaceutically acceptable salts;
1-Cyclopropyl-6-fluoro-7-(2-jydroxymethyl-6,7-dihydro-4H-thieno[3,2-c]pyridine-5-yl)-8-methoxy-4-oxo-1,4-dihydro-quinoline-3-carboxylic acid and its pharmaceutically acceptable salts;
1-Cyclopropyl-7-(2-formyl-6,7-dihydro-4H-thieno[3,2-c]pyridine-5-yl)-5,6,8-trifluoro-4-oxo-1,4-dihydro-quinoline-3-carboxy fluoroborate and its pharmaceutically acceptable salts;
1-Cyclopropyl-7-(2-nitro-6,7-dihydro-4H-thieno[3,2-c]pyridine-5-yl)-5,6,8-trifluoro-4-oxo-1,4-dihydro-quinoline-3-carboxy fluoroborate and its pharmaceutically acceptable salts;
1-Cyclopropyl-7-(2-nitro-6,7-dihydro-4H-thieno[3,2-c]pyridine-5-yl)-5,6,8-trifluoro-4-oxo-1,4-dihydro-quinoline-3-carboxylic acid and its pharmaceutically acceptable salts.

5. A composition comprising a compound of formula (I) as defined in claim 1 or a therapeutically acceptable salt thereof, and a therapeutically acceptable excipient.

6. A pharmaceutical composition, which comprises a compound as defined in claim 5, and a pharmaceutically acceptable carrier, diluents or excipients or solvate.

7. A pharmaceutical composition according to claim 5, in the form of tablets, pills, capsules, powder, granules, syrup, solution or suspension.

8. A method for treating infections comprising administering a therapeutically acceptable amount of compound of formula (I) as defined in claim 1, or a therapeutically acceptable salt thereof.

9. A method for treating an infection caused by gram-positive organisms, gram-negative organisms, mycobacterial infections or nosocomial infections comprising administering an effective amount of a compound according to claim 1 to a mammal in need thereof.

10. The method as claimed in claim 8 wherein the compound is administered orally, nasally, parenterally, topically, transdermally, or rectally.

11. Use of the compounds as claimed in claim 1 or their pharmaceutically acceptable salts for the preparation of medicine suitable for the treatment of infection caused by gram-positive organisms, gram-negative organisms, mycobacterial infections or nosocomial infections.

12. A process for the preparation of a compound of formula (I) as defined in claim 1, where all symbols are as defined earlier, and including their tautomeric forms, their stereoisomers, their pharmaceutically acceptable salts, their pharmaceutically acceptable solvates, which comprises:
i) converting a compound of formula (II) to compound of formula (III) 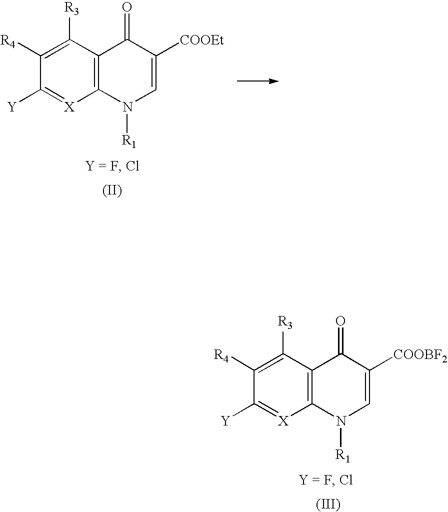
ii) reacting a compound of formula (III) with a compound of formula (IV) to obtain (V) 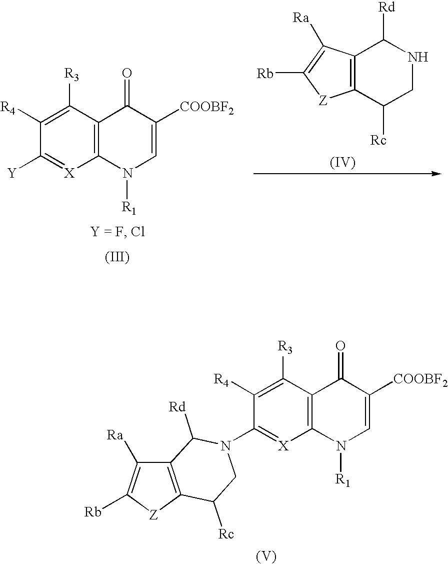
iii) converting the compound of formula (V) to compounds of formula (I) 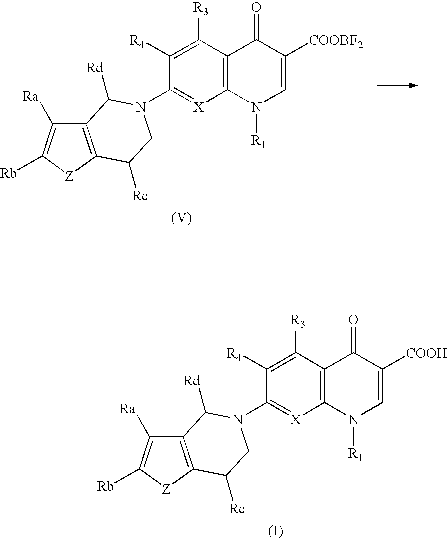
Where Ra, Rb, Rc, Rd, R₁, R₃ & R₄ are as described elsewhere in the specification and R₂=—OBF₂, —OH.
